(19) United States
(12) Patent Application Publication
(10) Pub. No.: US 2013/0018307 A1
(43) Pub. Date: Jan. 17, 2013

(54) Multi-Lumen Steerable Catheter

(71) Applicants: Carey Lee, Sunnyvale, CA (US); Nan Bai, Mountain View, CA (US); Jessica W. Ji, Fremont, CA (US); Yi Xue, East Palo Alto, CA (US); Lynn F. Peng, Stanford, CA (US)

(72) Inventors: Carey Lee, Sunnyvale, CA (US); Nan Bai, Mountain View, CA (US); Jessica W. Ji, Fremont, CA (US); Yi Xue, East Palo Alto, CA (US); Lynn F. Peng, Stanford, CA (US)

(21) Appl. No.: 13/548,814

(22) Filed: Jul. 13, 2012

Related U.S. Application Data
(60) Provisional application No. 61/572,448, filed on Jul. 15, 2011.

(51) Int. Cl. A61M 25/092

(52) U.S. Cl. 604/95.04

(57) ABSTRACT

A steerable multi-lumen catheter is provided which can be used in cardiology procedures where the catheter allows clinicians to access the small pulmonary arteries for monitoring and performing interventional procedures. The catheter allows precise changes in direction and simplifies challenging navigation procedures such as crossing a thick and calcified aortic valve with a wire. A bendable tip aids navigation during interventional radiology and neurology procedures to enter tortuous vessels or aneurysms.

### CROSS-REFERENCE TO RELATED APPLICATIONS

This application claims priority from U.S. Provisional Patent Application 61/572,448 filed Jul. 15, 2011, which is incorporated herein by reference.

### FIELD OF THE INVENTION

This invention relates to diagnostic and interventional procedures. In particular, the invention relates to steerable catheters.

### BACKGROUND OF THE INVENTION

Pediatric cardiologists often need to catheterize pulmonary and aortic vessels for diagnostic and interventional procedures. These small vessels often branch off at acute angles, requiring multiple time-consuming and complex catheter exchanges to access. The present invention advances the art and overcomes at least some of the shortcomings of current catheters used for these procedures.

### SUMMARY OF THE INVENTION

A steerable dual lumen catheter is provided having a flexible catheter with a proximal end, a distal end, and a distal portion with a pre-bend configuration. The distal portion in the pre-bend configuration can be bent over a range up to 180 degrees relative to the catheter's unbent and straightened configuration. A first lumen is present through the catheter and open at both the proximal and distal ends of the catheter. A second lumen is present through the catheter and open at the proximal end of the catheter, but closed at the distal end of the catheter. A straightening wire is movable inside the second lumen and able to change the pre-bend configuration of the distal portion over a range of 0 degrees when the catheter is straightened and the straightening wire is pushed-in towards the distal end of the second lumen to 180 degrees when the straightening wire is pulled-away from the distal portion and the catheter is in the pre-bend configuration. The straightening wire is inserted in the second lumen for manipulation of the pre-bend configuration of the distal portion simultaneously when a medical instrument or a guide wire has been inserted from the proximal end inside the first lumen and, where applicable, moved beyond the distal end of the catheter. The stiffness of the straightening wire can be uniform or non-uniform over its length.

In one example, the catheter does not have any open sides along the longitudinal wall of said catheter. The first and second lumen can have similar or different shapes, sizes and placements in the catheter. In one example, at least one or both of the cross-sections of the first lumen and of the second lumen is circular. In another example, at least one or both of the cross-sections of the first lumen and of the second lumen is not circular. In yet another example, the cross-section of the first lumen is larger than the cross-section of the second lumen. In still another example, the cross-section of the first and the cross-section of the second lumen are more or less equal. In still another example, the first (main) lumen is more or less centrally placed over the cross-section of the catheter. In still another example, there could be one or more lumens in addition to the first and second (dual) lumen. The one or more additional lumens could be open at both their respective proximal and distal ends or the one or more additional lumens could be open at their proximal ends, but closed at their distal ends potentially for additional straigthening wires.

### BRIEF DESCRIPTION OF THE DRAWINGS

FIGS. 1A-B show an example of straightening of a dual lumen catheter according to an exemplary embodiment of the invention. FIG. 1A shows a pre-bent configuration (straightening wire not inserted). FIG. 1B shows a straightened configuration (straightening wire inserted).

FIGS. 2A-B show example of a dual lumen catheter 200, 230 according to exemplary embodiments of the invention. FIG. 2A shows an example of a side view of a dual lumen catheter 200 with a first lumen 210 (dashed line indicating first lumen running through the catheter and open at proximal and distal end of the catheter) and a second lumen 220 (dashed line indicating second lumen running through the catheter and open only at proximal end of the catheter, but closed at the distal end of the catheter, i.e. dashed line stops before distal end of the catheter). FIG. 2B shows catheter 230 with straightening wire 240 partially inserted in the second lumen (note second lumen is not shown in FIG. 2B for clarity purposes) and medical instrument 250 simultaneously inserted in the first/main lumen (note first lumen is not shown in FIG. 2B for clarity purposes), whereby medical instrument 250 has passed through the distal end of the first lumen.

FIGS. 3A-C show a cross-section of the proximal ends of catheters (310, 320, 330—respectively a 4 Fr, 4 Fr and 7 Fr size) with first (main) lumen (respectively 312, 322, 332) and second distally closed (respectively 314, 324, 334) lumen according to exemplary embodiments of the invention.

### DETAILED DESCRIPTION

The present invention provides a catheter with a first central lumen and a controllable, bendable tip that could be quickly navigated into target vessels allowing e.g. cardiologist a quicker way to navigate to small arteries in the pulmonary system.

The dual lumen catheter according to an embodiment of the invention offers steerable tip functionality through a straightening wire system. The catheter, which is pre-shaped, roughly like a pigtail catheter, features a two-lumen design from the proximal to the distal end. A first lumen, which is open at both ends, is preserved as the first (main) lumen for guide wires, contrast injection, device deployment or other medical instruments. A second lumen, which is open at the proximal end but closed at the distal end, houses a stiffer straightening wire. The closed end prevents the straightening wire from passing through. In other words, the straightening wire is introduced from the proximal end of the catheter and can be pushed into, but not out of, the distal tip end. It is noted that the invention is not limited to a dual lumen, but can have more than 2 lumens as long as at least one lumen used for the straightening wire is open at the proximal end but closed at the distal end to maintain the functionality as described herein.

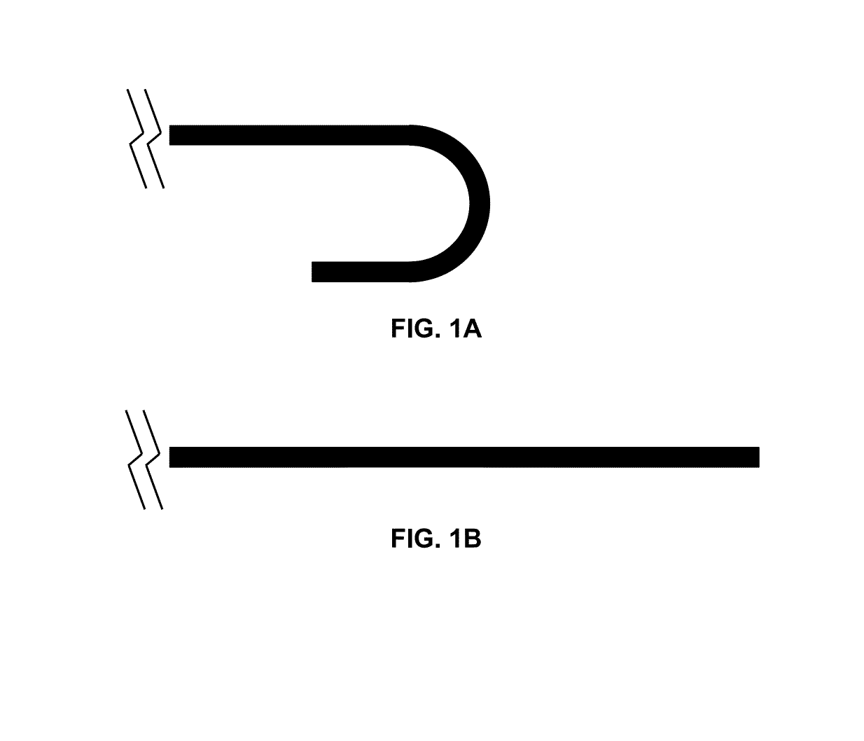

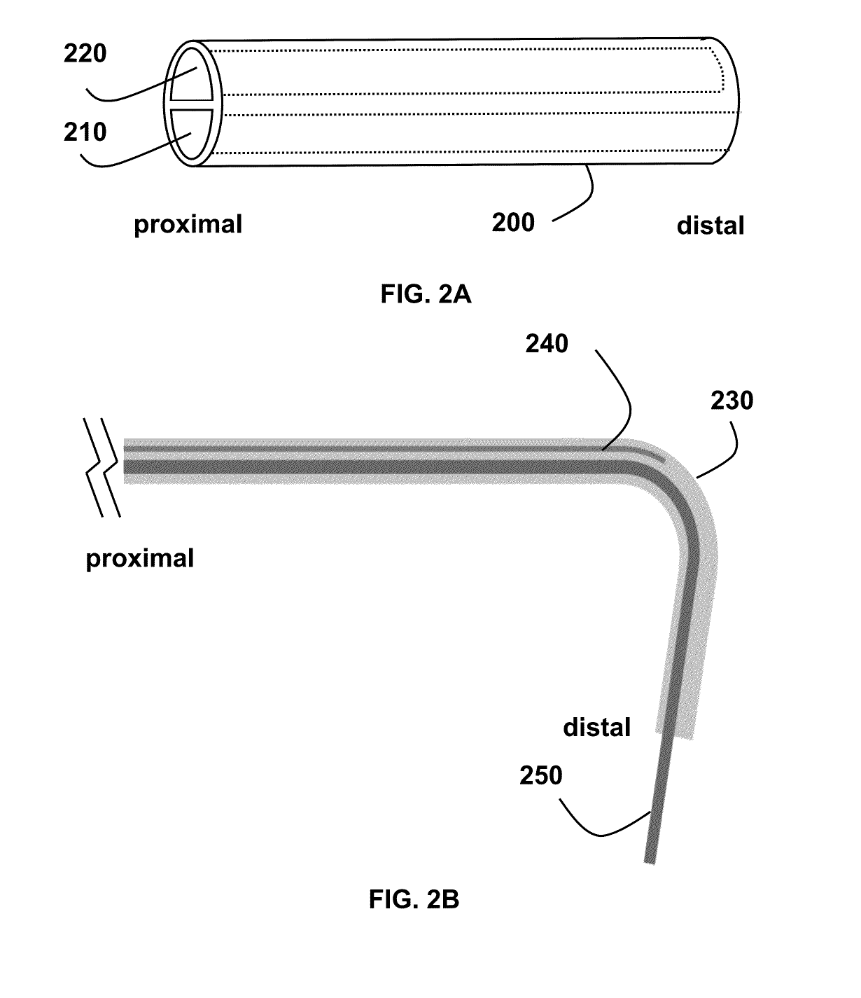

The operation of the device is entirely intuitive (FIGS. 1A-B and FIGS. 2A-B). The catheter straightens as the wire is pushed in. The catheter then returns to its original curved shape when the wire is pulled out. Different bend angles, varying from 0 to 180 degrees (relative to a straight catheter), can be achieved depending on how far the straightening wire is inserted. The straightening wire can be entirely removed and reinserted as desired. Additionally, straightening wires of different stiffnesses can be employed to change the stiffness of the catheter body.

The body of the catheter is unique from other steerable catheters in that it is a flexible material that can follow a guide wire into a vessel. Once the correct angle is obtained to enter a vessel with manipulation of the straightening wire, a guide wire can be extended through the first open lumen, past its distal open end and into a vessel. The catheter can then be advanced over the guide wire into a vessel, with or without the straightening wire still in place. This allows the same catheter to engage a vessel and to enter a vessel, thereby allowing the same catheter to perform diagnostic imaging as well as interventional procedures. The same catheter, once in the vessel, could then be used to enter other side-branches of the vessel.

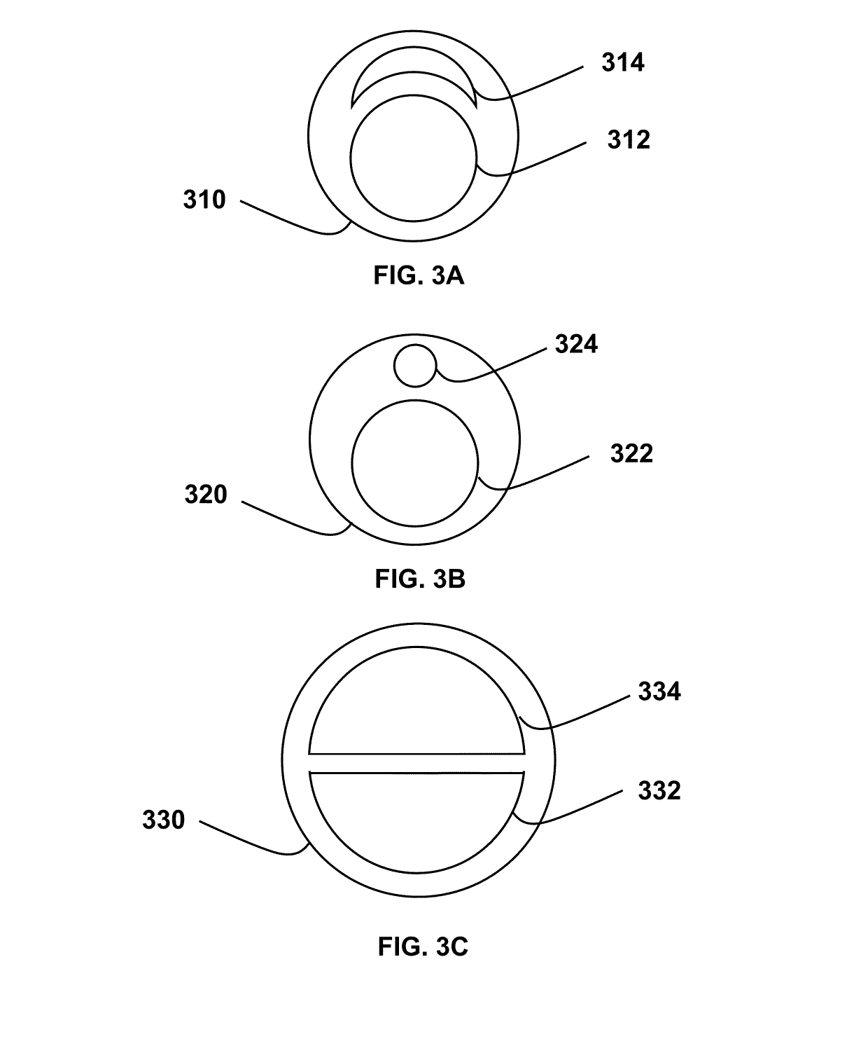

FIGS. 3A-C depict some exemplary configurations of dual lumens ranging from a wide variety of cross-sectional configurations to enable a small overall catheter diameter. In one example, a first (main) lumen of at least 0.035 inches was developed so that the guide wires for balloon catheters can pass through. In another example, the outer diameter was minimized so that cardiologists can access very small vessels. In one example, the catheter was as small as 4 Fr, which is approximately 0.053 inches.

The secondary lumen is used to accommodate a straightening wire, which can have variable diameter or stiffness to accommodate different needs. The straightening wire can be of uniform stiffness or have a floppy tip (e.g. non-uniform stiffness) to help it follow the bend of the catheter without damaging the walls. An example of the diameter of the straightening wire is 0.010 inches.

The device can be used in cardiology procedures where the steerable catheter allows clinicians to access the small pulmonary arteries for monitoring and performing interventional procedures. It can also be used in other disease states, such as pulmonary vein stenosis, critical aortic stenosis, or critical pulmonary valve stenosis, to enter different cardiac chambers. In addition, it also has potential viability in other clinical fields. Cardiologists who work with adults have difficulty finding the small orifice of the aortic valve in patients with aortic valve stenosis. A catheter that allows small changes in direction would simplify the process of crossing a thick and calcified aortic valve with a wire. A bendable tip can also aid navigation during interventional radiology and neurology procedures to enter tortuous vessels or aneurysms. In short, many procedures utilize small catheters and would benefit from a simple way of directing the tip.

An advantage of the device is that it can be easily operated by a single cardiologist working alone. Optionally, if another set of hands is desired and available, the straightening wire in the second lumen and the device in the first lumen are entirely independent and physically separate, so control of either can be easily delegated.

In conclusion, the present invention provides a catheter where the tip has a variable bend angle of 0 to 180 degrees via a straightening wire/dual lumen mechanism. The catheter has a dedicated secondary lumen for the straightening wire, keeping first (main/central) lumen available for guide wires/ diagnostics/interventions. Selective stiffening of the catheter body via inserting straightening wires of different stiffnesses can be employed. The catheter has the ability to follow a guide wire into a vessel after the vessel mouth has been engaged and to continue to gain access into further branches of the vessel. The tip curvature can be varied, and the wire stiffness can be changed to suit different clinical needs. Lumen geometry and overall scale of the catheter can also be varied.

1. A steerable multi-lumen catheter, comprising:
(a) a flexible catheter with a proximal end, a distal end, and a distal portion with a pre-bend configuration, wherein said distal portion in said pre-bend configuration is bent over a range up to 180 degrees relative to said catheter's unbent and straightened configuration;
(b) a first lumen through said catheter and open at both said proximal and said distal ends of said catheter;
(c) a second lumen through said catheter and open at said proximal end of said catheter, but closed at said distal end of said catheter;
(d) a straightening wire movable inside said second lumen and able to change said pre-bend configuration of said distal portion over a range of 0 degrees when said catheter is straightened and said straightening wire is pushed-in towards said distal end of said second lumen to 180 degrees when said straightening wire is pulled-away from said distal portion and said catheter is in said pre-bend configuration,
whereby simultaneously allowing (i) said straightening wire inserted in said second lumen for manipulation of said pre-bend configuration of said distal portion and (ii) a medical instrument or a guide wire inserted from said proximal end inside said first lumen and where applicable moved beyond said distal end.

2. The steerable multi-lumen catheter as set forth in claim 1, wherein said catheter does not have any open sides along the longitudinal wall of said catheter.

3. The steerable multi-lumen catheter as set forth in claim 1, wherein the cross-section of said first lumen and/or of said second lumen is circular or not circular.

4. The steerable multi-lumen catheter as set forth in claim 1, wherein the cross-section of said first lumen is larger than the cross-section of said second lumen.

5. The steerable multi-lumen catheter as set forth in claim 1, wherein the cross-section of said first and the cross-section of said second lumen are more or less equal.

6. The steerable multi-lumen catheter as set forth in claim 1, wherein said first lumen is more or less centrally placed over the cross-section of said multi-lumen catheter.

7. The steerable multi-lumen catheter as set forth in claim 1, wherein the stiffness of said straightening wire is uniform or non-uniform.

8. The steerable multi-lumen catheter as set forth in claim 1, further comprising one or more lumens in addition to said first lumen and said second lumen, wherein said one or more additional lumens are open at their respective proximal and distal ends.

9. The steerable multi-lumen catheter as set forth in claim 1, further comprising one or more lumens in addition to said first lumen and said second lumen, wherein said one or more additional lumens are open at their proximal ends, but closed at their distal ends.
